(19) United States
(12) Patent Application Publication
(10) Pub. No.: US 2022/0016231 A1
(43) Pub. Date: Jan. 20, 2022

(54) HEPATITIS B VIRUS VACCINE AND USES THEREOF

(71) Applicant: JRHS Therapeutics Inc., Beijing (CN)

(72) Inventors: Zhuang SU, Andover, MA (US); Meijia YANG, Scituate, MA (US)

(21) Appl. No.: 17/413,026

(22) PCT Filed: Dec. 9, 2019
(86) PCT No.: PCT/US19/65151

§ 371 Date: Jun. 11, 2021

Related U.S. Application Data
(60) Provisional application No. 62/778,549, filed on Dec. 12, 2018.

(51) Int. Cl. A61K 39/12; C07K 14/005

(52) U.S. Cl. CPC A61K 39/12; C07K 14/005; C12N 2730/10123; C12N 2730/10152; C12N 2730/10171; C12N 2730/10122; C12N 2730/10134

(57) ABSTRACT

A hepatitis B vims (HBV) vaccine particle is described, including a recombinant HBV surface antigen including L surface protein; optionally M surface protein; and optionally S surface protein; wherein the molar percentage of L surface protein to the sum of L, M, and S surface proteins is at least about 1 mole %, 8 mole %, 10 mole %, 20 mole %, 30 mole %, 40 mole %, or 50 mole %. Methods of making the same and methods of treating or preventing HBV infection in a subject using the same are also described.

### CROSS REFERENCE TO RELATED APPLICATIONS

This application claims benefit of U.S. Provisional Application No. 62/778,549, filed Dec. 12, 2018, the entire contents of which are hereby incorporated by reference.

### INCORPORATION BY REFERENCE

The entire contents of all patents, patent applications, and articles cited herein are expressly incorporated by reference in its entirety.

### FIELD OF THE INVENTION

The invention relates to hepatitis B virus (HBV) vaccines.

### BACKGROUND OF THE INVENTION

Chronic hepatitis B virus (HBV) infections continue to be a challenge for the global public health care management. Worldwide over 350 million of patients are affected by the chronic hepatitis B infections, resulting in over 1 M deaths each year. See, e.g., Kane, M., 1995. Global programme for control of hepatitis B infection. Vaccine, 13, pp. S47-S49; Lavanchy, D., 2004, Hepatitis B virus epidemiology, disease burden, treatment, and current and emerging prevention and control measures. Journal of Viral Hepatitis, 11(2), pp. 97-107; and Maddrey, W. C., 2001. Hepatitis B—an important public health issue, Clinical laboratory, 47(1-2), pp. 51-55. Particularly in developing country such as in China, nearly 10% of the population are affected (see, e.g., Yao, J. L., 1996, Perinatal transmission of hepatitis B virus infection and vaccination in China. Gut, 38 (Suppl 2), pp. S37-S38; Liang, X., et al., 2009, Epidemiological serosurvey of hepatitis B in China—declining HBV prevalence due to hepatitis B vaccination. Vaccine, 27(47), pp. 6550-6557). Additionally, a large percentage of the chronically ill carrier patients will develop hepatocellular carcinoma. See, e.g., Bosch F. X., Ribes, J. and Borràs, J., 1999. Epidemiology of primary liver cancer, in Seminars in Liver Disease, Vol. 19, No. 03, pp. 271-285, Thieme Medical Publishers, Inc.; Bosch, F. X., Ribes, J., Cléries, R. and Diaz, M., 2005, Epidemiology of hepatocellular carcinoma, Clinics in Liver Disease, 9(2), pp. 191-211; Ribes, J., Clèries, R., Rubió, A., Hernández, J. M., Mazzara, R., Madoz, P., Casanovas, T., Casanova, A., Gallen, M. Rodriguez, C. and Moreno, V., 2006, Cofactors associated with liver disease mortality in an HBsAg-positive Mediterranean cohort: 20 years of follow-up, International Journal of Cancer, 119(3), pp. 687-694.

Current treatment such as using nucleoside inhibitors do not inhibit the transcription of covalently closed circular DNA (cccDNA) and often leads to drug resistance. Interferon alpha treatment leads to intolerable side effects (Lok, A. S. and McMahon, B. J., 2007. Chronic hepatitis B. Hepatology, 45(2), pp. 507-539)) and at best only 10% of the treated patients achieve seroconversions. Therefore, a more effective treatment option is still needed to address this current health care issue.

The surface envelop of the hepatitis B virus contains three proteins named as L, M, and S. The three proteins share common C-terminus, while the M-form contains an extra, N-terminal PreS2 sequence compared to S, and the L-form contains an additional PreS1 sequence compared to M and S (Ganem, D. and Varmus, H. E., 1987. The molecular biology of the hepatitis B viruses. Annual Review of Biochemistry, 56(1), pp. 651-693), The PreS1 sequence reportedly contains a receptor binding sequence (aa 21-47) and is responsible for the specific binding of the virus to the liver cells. See, e.g., Barrera, A., Guerra, B., Notvall, L. and Lanford, R. E., 2005, Mapping of the hepatitis B virus pre-S1 domain involved in receptor recognition, Journal of Virology, 79(15), pp. 9786-9798; Neurath, A. R., Kent, S. B. H., Strick, N. and Parker, K., 1986, Identification and chemical synthesis of a host cell receptor binding site on hepatitis B virus, Cell, 46(3), pp. 429-436; Neurath, A. R., Seto, B. and Strick, N., 1989, Antibodies to synthetic peptides from the preS1 region of the hepatitis B virus (HBV) envelope (env) protein are virus-neutralizing and protective, Vaccine, 7(3), pp. 234-236; and Dash, S., Rao, K. V. and Panda, S. K., 1992, Receptor for pre-S1 (21-47) component of hepatitis B virus on the liver cell: Role in virus cell interaction, Journal of Medical Virology, 37(2), pp. 116-121. Recently, sodium taurocholate cotransporting polypeptide has been identified as a functional receptor for human hepatitis B virus. See, e.g., Yan, H., et al., 2012, Sodium taurocholate cotransporting polypeptide is a functional receptor for human hepatitis B and D virus. Elife, 1, p.e00049; Ni, Y., et al., 2014, Hepatitis B and D viruses exploit sodium taurocholate co-transporting polypeptide for species-specific entry into hepatocytes. Gastroenterology, 146(4), pp. 1070-1083.

There remains a need for effective HBV vaccines.

### SUMMARY OF THE INVENTION

In one aspect, a hepatitis B virus (HBV) vaccine particle is disclosed, comprising a recombinant HBV surface antigen comprising:

L surface protein;

optionally M surface protein; and

optionally S surface protein;

wherein the percentage of L surface protein in the L, M, and S surface proteins is at least about 1 mole %.

In any one of the embodiments disclosed herein, the percentage of L surface protein in the L, M, and S surface proteins is at least about 2 mole %, 3 mole %, 4 mole %, 5 mole %, 6 mole %, 7 mole %, or 8 mole %.

In any one of the embodiments disclosed herein, the percentage of L surface protein in the L, M, and S surface proteins is more than about 8 mole %.

In any one of the embodiments disclosed herein, the percentage of L surface protein in the L, M, and S surface proteins is more than about 9 mole %, 10 mole %, 11 mole %, 12 mole %, 13 mole %, 14 mole %, 15 mole %, 16 mole %, 17 mole %, 18 mole %, 19 mole %, 20 mole %, 21 mole %, 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, or 50 mole %.

In any one of the embodiments disclosed herein, the percentage of L surface protein in the L, M, and S surface proteins is at least about 60 mole %, 70 mole %, 80 mole %, 90 mole %, or 100 mole %.

In any one of the embodiments disclosed herein, the HBV vaccine particle does not include M or S protein.

In any one of the embodiments disclosed herein, the HBV vaccine particle is a virus-like-particle.

In any one of the embodiments disclosed herein, the percentage of L surface protein in the L, M, and S surface proteins is from about 10 mole % to about 40 mole %, 5-15 mole %, 15-25 mole %, 25-40 mole %, or 40-60 mole %.

In any one of the embodiments disclosed herein, the HBV vaccine particle includes clone A4 or 51 as shown in FIG. 9.

In any one of the embodiments disclosed herein, the L surface protein is encoded by a recombinant nucleic acid sequence which does not have an internal cis-element. In some embodiments, the L surface protein is encoded by a recombinant nucleic acid sequence which does not have an internal cis-element driving S protein expression. In some embodiments, the L surface protein is encoded by a recombinant nucleic acid sequence which does not have an internal cis-element driving M protein expression. In some embodiments, the L surface protein is encoded by a recombinant nucleic acid sequence which does not have an internal cis-element driving M or S protein expression.

In another aspect, a HBV vaccine is disclosed, including the HBV vaccine particle of any one of the embodiments and an adjuvant.

In any one of the embodiments disclosed herein, the adjuvant is selected from the group consisting of alum, a toll-like receptor, and colloidal gold.

In yet another aspect, a method of treating or preventing HBV infection in a subject in need thereof is disclosed, comprising administering to the subject an effective amount of the HBV vaccine of any one of the embodiments disclosed herein.

In any one of the embodiments disclosed herein, the subject is human.

In yet another aspect, a recombinant nucleic acid sequence encoding L surface protein is disclosed, wherein the recombinant nucleic acid sequence does not have an internal cis-element.

In yet another aspect, a recombinant expression vector for expressing L surface protein is disclosed, comprising the recombinant nucleic acid sequence of any one of the embodiments disclosed herein.

In yet another aspect, a cell is described, wherein the cell is transformed with the recombinant expression vector of any one of the embodiments disclosed herein.

In any one of the embodiments disclosed herein, the cell is additionally transformed by

a second recombinant expression vector comprising a second recombinant nucleic acid sequence encoding the S surface protein, and

a third recombinant expression vector comprising a third recombinant nucleic acid sequence encoding the M surface protein.

In any one of the embodiments disclosed herein, the cell is additionally transformed by one or more additional recombinant expression vectors.

In any one of the embodiments disclosed herein, the cell is additionally transformed by a fourth expression vector comprising a fourth recombinant nucleic acid sequence encoding the HBV core antigen.

In any one of the embodiments disclosed herein, the cell is derived from an insect or mammalian protein expression host. In any one of the embodiments disclosed herein, the cells are derived from E coli or fungus.

In any one of the embodiments disclosed herein, the cell is derived from a HEK-293 cell or a CHO cell.

In yet another aspect, a method for preparing an HBV vaccine particle is described, comprising:

a) providing recombinant expression vectors comprising a first, second, and third recombinant nucleic acid sequences encoding L, M, and S surface proteins respectively; and wherein the first, second, and third recombinant nucleic acid sequences do not have an internal cis-element;

b) transforming cells with the recombinant expression vectors; and

c) culturing and selecting for cells to co-express L, M, and S surface proteins.

In any one of the embodiments disclosed herein, each of L, S, and M surface proteins is in a separate expression vector.

In any one of the embodiments disclosed herein, the method further includes selecting for cells to express L surface protein in a percentage of at least about 1 mole %, 2 mole %, 3 mole %, 4 mole %, 5 mole %, 6 mole %, 7 mole %, 8 mole %, 9 mole %, 10 mole %, 11 mole %, 12 mole %, 13 mole %, 14 mole %, 15 mole %, 16 mole %, 17 mole %, 18 mole %, 19 mole %, 20 mole %, 21 mole %, 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, or 50 mole % in the L, M, and S surface proteins.

In any one of the embodiments disclosed herein, the method further includes selecting for cells to express L surface protein in a percentage of at least about 60 mole %, 70 mole %, 80 mole %, 90 mole %, or 100 mole % in the L, M, and S surface proteins.

In any one of the embodiments disclosed herein, the recombinant expression vectors further comprise a fourth recombinant nucleic acid sequence encoding the HBV core antigen; and step c) comprises culturing and selecting for cells to co-express L, M, and S surface proteins and HBV core antigen.

In any one of the embodiments disclosed herein, the cells are derived from an insect, or mammalian protein expression host. In any one of the embodiments disclosed herein, the cells are derived from E coli or fungus. In any one of the embodiments disclosed herein, the cells are derived from HEK-293 cells or CHO cells.

Any aspect or embodiment disclosed herein may be combined with another aspect or embodiment disclosed herein. The combination of one or more embodiments described herein with other one or more embodiments described herein is expressly contemplated.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods, and materials are described below. All publications, patent applications, patents, and other references mentioned herein are incorporated by reference in their entirety. In addition, the materials, methods, and examples are illustrative only, and not intended to be limiting.

### DESCRIPTION OF THE DRAWINGS

The invention is described with reference to the following figures, which are presented for the purpose of illustration only and are not intended to be limiting. In the drawings:

FIG. 1 depicts Western blot analysis of L protein expression: lane 1. molecular weight ladder; lane 2. Mock transfection; lane 3. L form; lane 4. L form with additional N-terminal, signal peptide. Note the secreted L protein is visualized as 49 KDa-60 KDa proteins.

FIG. 2 shows transient expression of S, M, and L proteins, either alone or in combinations (presence of the HBsAg particles in the conditioned medium were detected by ELISA analysis; co-transfection S+M+L showed low level of HBsAg particles but is detectable, confirming that high expression of L protein inhibits secretion of M or S proteins).

FIG. 3 shows ELISA detection of transient expression of L, M, and S proteins in presence of the core proteins, indicating that core proteins do not affect secretion of the particles.

FIG. 4A shows Western blot screening of 293F stable clones 16, 23, 50, 51, and 12 that expressed proteins recognized by anti PreS2 monoclonal antibodies S26 (left panel) and anti S polyclonal antibodies (right panel). Conditioned medium was harvested after 72 hours and presence of L, M, and S forms was screened by Western analysis using PreS2 specific monoclonal antibody S26 (Left panel) and polyclonal antibodies against S (Right panel).

FIG. 4B shows Western blot screening of CHO stable clones 7C8 and 10E3 that expressed proteins recognized by anti PreS2 monoclonal antibodies S26 (left panel) and anti S polyclonal antibodies (right panel).

FIG. 5 shows Western blot screening of 293F stable clones A4 and 51 that expressed proteins recognized by anti S polyclonal antibodies (left panel) and anti PreS2 monoclonal antibodies S26 (right panel). Conditioned medium was harvested after 72 hours and presence of L, M, and S forms was screened by Western analysis.

FIG. 6 shows additional stable expression clones 26, 43, 88 were screened for presence of L, M, and S proteins. Individual clones were grown in 293 FreeStyle expression medium. Conditioned medium were harvested after 72 hours and presence of L, M, and S forms were screened by Western blot analysis using PreS2 specific monoclonal antibody S26 (left panel) and polyclonal antibodies against S (right panel).

FIG. 7 shows the silver staining analysis fractions of the final SEC purification for clone 51. Conditioned medium of clone 51 were concentrated, captured by hydroxy apatite and fractioned by size exclusion chromatography. The last two lanes are BSA protein fractionated as reference proteins.

FIG. 8 shows the silver staining analysis fractions of the final SEC purification for clone A4. Conditioned medium of clone A4 were concentrated, HBsAg particles were captured by hydroxy apatite and fractionated by size exclusion chromatography.

FIG. 9 shows Coomassie staining of purified HBsAg proteins from clones A4 and 51. Approximately 10 μg protein by BCA estimation were loaded to each lane. Protein identities were verified by Western blot and mass spectroscopy after in gel peptide. Lanes 1, 2, 3 are three different preparations of the clone A4. Lane 4 is one of the representative preparations of the clone 51.

FIG. 10 shows Western blot analysis of purified HBsAg particles using anti-S polyclonal antibodies (left panel), and anti-PreS2 monoclonal antibody S26 (middle panel), and anti-PreS1 monoclonal antibody AP1 (right panel). The left and middle lanes are two different preparations of the clone A4. The right lane is a purified protein preparation from clone 51.

FIG. 11 shows that the L, M, and S forms are at least partially glycosylated. Purified proteins are treated with PNGase and removal of glycans were monitored by SDS PAGE and Western blot analysis.

FIG. 12 shows electron microscopy of purified HBsAg particles from clone 16.

FIG. 13 shows electron microscopy of the purified HBsAg particles from clone 51.

FIG. 14 shows the mouse serum titer where two mice were immunized using purified LMS virus-like particles. Yeast-derived HBsAg was used as an immunization control. 35 days after the initial immunization, the titers of the antibody response against the virus-like particles were determined by serial dilutions. HBsAg-1, HBsAg-2: mice were immunized using S-form HBsAg produced in yeast. #16-1, #16-2: mice were immunized with purified LMS HBsAg derived from clone 16. #51-1, #51-2: mice were immunized with purified LMS HBsAg derived from clone #51.

FIG. 15 shows antibody titers assayed by using purified HBsAg particles. Each bar represents the antibody titer in one mouse of the Balb C strain.

FIG. 16 shows antibody responses against the PreS2 region were tested using purified, linear PreS2 peptide. Sera from all four mice were reactive against the PreS2 peptide, but not the BSA control.

FIG. 17 shows spleens from all four immunized mice were removed, and hybridomas were generated using B cells from the mice. Clones that were reactive against the purified HBsAg particles were grouped in S, PreS2, PreS1, or unknown bins.

### DETAILED DESCRIPTION OF THE INVENTION

### HBV Vaccines

In one aspect, a hepatitis B virus (HBV) vaccine particle is disclosed, comprising a recombinant HBV surface antigen comprising:

L surface protein;

optionally M surface protein; and

optionally S surface protein;

wherein the percentage of L surface protein in the L, M, and S surface proteins is at least about 1 mole %.

In some embodiments, the percentage of L surface protein in the L, M, and S surface proteins is at least about 2 mole %, 3 mole %, 4 mole %, 5 mole %, 6 mole %, 7 mole %, or 8 mole %.

In some embodiments, the percentage of L surface protein in the L, M, and S surface proteins is more than about 8 mole %.

In some embodiments, the percentage of L surface protein in the L, M, and S surface proteins is more than about 9 mole % or 10 mole %. In some embodiments, the percentage of L surface protein in the L, M, and S surface proteins is more than about 9 mole %, 10 mole %, 11 mole %, 12 mole %, 13 mole %, 14 mole %, 15 mole %, 16 mole %, 17 mole %, 18 mole %, 19 mole %, 20 mole %, 21 mole %, 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, or 50 mole %, or in a ranged bound by any two values disclosed herein.

In some embodiments, the percentage of L surface protein in the L, M, and S surface proteins is at least about 15 mole %, 20 mole %, 25 mole %, 30 mole %, or 40 mole %. In some embodiments, the percentage of L surface protein in the L, M, and S surface proteins is at least about 60 mole %, 70 mole %, 80 mole %, 90 mole %, or 100 mole %. In some embodiments, the HBV vaccine particle does not include M or S protein. In some embodiments, the HBV vaccine particle is a virus-like-particle.

In some embodiments, the percentage of L surface protein in the L, M, and S surface proteins is from about 10 mole % to about 40 mole %. In some embodiments, the percentage of L surface protein in the L, M, and S surface proteins is from about 5-15 mole %, 15-25 mole %, 25-40 mole %, or 40-60 mole %. In some embodiments, the percentage of L surface protein in the L, M, and S surface proteins is from about 1 mole % to about 40 mole %. In some embodiments, the percentage of L surface protein in the L, M, and S surface proteins is from about 2 mole % to about 40 mole %. In some embodiments, the percentage of L surface protein in the L, M, and S surface proteins is from about 4 mole % to about 40 mole %. In some embodiments, the percentage of L surface protein in the L, M, and S surface proteins is from about 5 mole % to about 40 mole %. In some embodiments, the percentage of L surface protein in the L, M, and S surface proteins is from about 6 mole % to about 40 mole %. In some embodiments, the percentage of L surface protein in the L, M, and S surface proteins is from about 7 mole % to about 40 mole %.

In some embodiments, the percentage of L surface protein in the L, M, and S surface proteins is from about 8 mole % to about 40 mole %. In some embodiments, the percentage of L surface protein in the L, M, and S surface proteins is from about 9 mole % to about 40 mole %. In some embodiments, the percentage of L surface protein in the L, M, and S surface proteins is from about 10 mole % to about 40 mole %. In some embodiments, the percentage of L surface protein in the L, M, and S surface proteins is from about 15 mole % to about 40 mole %. In some embodiments, the percentage of L surface protein in the L, M, and S surface proteins is from about 20 mole % to about 40 mole %. In some embodiments, the percentage of L surface protein in the L, M, and S surface proteins is from about 25 mole % to about 40 mole %. In some embodiments, the percentage of L surface protein in the L, M, and S surface proteins is from about 30 mole % to about 40 mole %.

In any one of the embodiments disclosed herein, the L surface protein is encoded by a recombinant nucleic acid sequence which does not have an internal cis-element. Applicants have surprisingly found that by removing the cis-element, more than certain percentage of the L surface protein (e.g., more than 8 mole % or 10 mole %) can be expressed. In any one of the embodiments disclosed herein, the internal cis-elements include promoters for transcription initiation of M and/or S forms. Thus, in some embodiments, the L surface protein is encoded by a recombinant nucleic acid sequence which does not have an internal cis-element driving S protein expression. In some embodiments, the L surface protein is encoded by a recombinant nucleic acid sequence which does not have an internal cis-element driving M protein expression. In some embodiments, the L surface protein is encoded by a recombinant nucleic acid sequence which does not have an internal cis-element driving M or S protein expression.

In another aspect, a HBV vaccine is disclosed, including the HBV vaccine particle of any one of the embodiments and an adjuvant. Any adjuvant able to stimulate and/or enhance an immune response is contemplated. Non-limiting examples of adjuvant includes alum, a toll-like receptor, and colloidal gold.

In yet another aspect, a method of treating or preventing HBV infection in a subject in need thereof is disclosed, comprising administering to the subject an effective amount of the HBV vaccine of any one of the embodiments disclosed herein. Non-limiting examples of subjects include human, monkey, cow, horse, dog, cat, and other mammals.

In any one of the embodiments disclosed herein, the subject is human.

In yet another aspect, a recombinant nucleic acid sequence encoding L surface protein is disclosed, wherein the recombinant nucleic acid sequence does not have an internal cis-element.

In yet another aspect, a recombinant expression vector for expressing L surface protein is disclosed, comprising the recombinant nucleic acid sequence of any one of the embodiments disclosed herein.

In yet another aspect, a cell is described, wherein the cell is transformed with the recombinant expression vector of any one of the embodiments disclosed herein. Non-limiting examples of cells include CHO and HEK-293 cells.

In any one of the embodiments disclosed herein, the cell is additionally transformed by

a second recombinant expression vector comprising a second recombinant nucleic acid sequence encoding the S surface protein, and

a third recombinant expression vector comprising a third recombinant nucleic acid sequence encoding the M surface protein.

In any one of the embodiments disclosed herein, the cell is additionally transformed by a fourth expression vector comprising a fourth recombinant nucleic acid sequence encoding the HBV core antigen. In any one of the embodiments disclosed herein, the cell is additionally transformed by one or more additional recombinant expression vectors.

In any one of the embodiments disclosed herein, the cell is derived from an insect or mammalian protein expression host, e.g., a HEK-293 cell or a CHO cell. In any one of the embodiments disclosed herein, the cells are derived from E coli or fungus.

### Methods of Preparation

In yet another aspect, a method for preparing an HBV vaccine particle is described, comprising:

a) providing recombinant expression vectors comprising a first, second, and third recombinant nucleic acid sequence encoding L, M, and S surface proteins respectively; and wherein the first, second, and third recombinant nucleic acid sequences do not have an internal cis-element;

b) transforming cells with the recombinant expression vectors; and

c) culturing and selecting for cells to co-express L, M, and S surface proteins.

In any one of the embodiments disclosed herein, each of L, M, and S surface proteins is in a separate expression vector.

In any one of the embodiments disclosed herein, the method further includes selecting for cells to express L surface protein in a percentage of at least about 1 mole %, 2 mole %, 3 mole %, 4 mole %, 5 mole %, 6 mole %, 7 mole %, 8 mole %, 9 mole %, 10 mole %, 11 mole %, 12 mole %, 13 mole %, 14 mole %, 15 mole %, 16 mole %, 17 mole %, 18 mole %, 19 mole %, 20 mole %, 21 mole %, 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, or 50 mole % in the L, M, and S surface proteins, or in a range bound by any two values disclosed herein.

In any one of the embodiments disclosed herein, the method further includes selecting for cells to express L surface protein in a percentage of at least about 15 mole %, 20 mole %, 25 mole %, 30 mole %, 40 mole %, 50 mole %, 60 mole %, 70 mole %, 80 mole %, 90 mole %, or 100 mole % in the L, M, and S surface proteins.

In any one of the embodiments disclosed herein, the recombinant expression vectors further comprise a fourth recombinant nucleic acid sequence encoding the HBV core antigen; and step c) comprises culturing and selecting for cells to co-express L, M, and S surface proteins and HBV core antigen.

In any one of the embodiments disclosed herein, the cells are derived from an insect or mammalian protein expression host, e.g., HEK-293 cells or CHO cells. In any one of the embodiments disclosed herein, the cells are derived from E coli or fungus.

### Pharmaceutical Compositions

This invention also provides a pharmaceutical composition comprising at least one of the HBV vaccine particle or HBV vaccine as described herein or a pharmaceutically acceptable salt or solvate thereof, and a pharmaceutically acceptable carrier.

The phrase “adjuvant” as used herein refers to any adjuvant known in the art.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as butylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers, and lubricants, such as sodium lauryl sulfate, magnesium stearate, and polyethylene oxide-polybutylene oxide copolymer as well as coloring agents, release agents, coating agents, sweetening, flavoring and perfuming agents, preservatives, and antioxidants can also be present in the compositions.

Formulations of the present invention include those suitable for oral, nasal, topical (including buccal and sublingual), rectal, vaginal, and/or parenteral administration. The formulations may conveniently be presented in unit dosage form and may be prepared by any methods well known in the art of pharmacy. The amount of active ingredient, which can be combined with a carrier material to produce a single dosage form, will vary depending upon the host being treated and the particular mode of administration. The amount of active ingredient, which can be combined with a carrier material to produce a single dosage form will generally be that amount of the HBV vaccine particle or HBV vaccine which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a HBV vaccine particle or HBV vaccine of the present invention with the carrier and, optionally, one or more accessory ingredients. In general, the formulations are prepared by uniformly and intimately bringing into association a HBV vaccine particle or HBV vaccine of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a HBV vaccine particle or HBV vaccine of the present invention as an active ingredient. A HBV vaccine particle or HBV vaccine of the present invention may also be administered as a bolus, electuary, or paste.

In solid dosage forms of the invention for oral administration (capsules, tablets, pills, dragees, powders, granules, and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose, and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active, or dispersing agent. Molded tablets may be made by molding, in a suitable machine, a mixture of the powdered compound moistened with an inert liquid diluent.

The tablets, and other solid dosage forms of the pharmaceutical compositions of the present invention, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying butortions to provide the desired release profile, other polymer matrices, liposomes, and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples are embedding compositions, which can be used including polymeric substances and waxes. The active ingredient can also be in micro-encapsulated form, if appropriate, with one or more of the above-described excipients.

Besides inert diluents, the oral compositions can also include adjuvants such as wetting agents, emulsifying and suspending agents, sweetening, flavoring, coloring, perfuming, and preservative agents.

Suspensions may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more HBV vaccine particle or HBV vaccine of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active pharmaceutical agents of the invention.

Formulations of the present invention which are suitable for vaginal administration also include pessaries, tampons, creams, gels, pastes, foams, or spray formulations containing such carriers as are known in the art to be apbutriate.

Powders and sprays can contain excipients such as lactose, talc, silicic acid, aluminum hydroxide, calcium silicates, and polyamide powder, or mixtures of these substances. Sprays can additionally contain customary butellants, such as chlorofluorohydrocarbons and volatile unsubstituted hydrocarbons, such as butane and butane.

Ophthalmic formulations, eye ointments, powders, solutions, and the like, are also contemplated as being within the scope of this invention.

Pharmaceutical compositions of this invention suitable for parenteral administration comprise one or more HBV vaccine particles or HBV vaccines of the invention in combination with one or more pharmaceutically acceptable sterile isotonic aqueous or nonaqueous solutions, dispersions, suspensions, or emulsions, or sterile powders which may be reconstituted into sterile injectable solutions or dispersions just prior to use, which may contain antioxidants, buffers, bacteriostats, solutes which render the formulation isotonic with the blood of the intended recipient, or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. One strategy for depot injections includes the use of polyethylene oxide-polybutylene oxide copolymers, wherein the vehicle is fluid at room temperature and solidifies at body temperature.

Injectable depot forms are made by forming microencapsule matrices of the subject HBV vaccine particle or HBV vaccine in biodegradable polymers such as polylactide-polyglycolide. Depending on the ratio of drug to polymer, and the nature of the particular polymer employed, the rate of drug release can be controlled. Examples of other biodegradable polymers include poly (orthoesters) and poly (anhydrides). Depot injectable formulations are also prepared by entrapping the drug in liposomes or microemulsions, which are compatible with body tissue.

When the HBV vaccine particle or HBV vaccine of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The HBV vaccine particle or HBV vaccine and pharmaceutical compositions of the present invention can be employed in combination therapies, that is, the HBV vaccine particle or HBV vaccine and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the HBV vaccine particle or HBV vaccine of the present invention may be administered concurrently with another anti-HBV agent), or they may achieve different effects (e.g., control of any adverse effects).

The HBV vaccine particle or HBV vaccine of the invention may be administered intravenously, intramuscularly, intraperitoneally, subcutaneously, topically, orally, or by other acceptable means. In some specific embodiments, the HBV vaccine particle or HBV vaccine disclosed herein is administered by nasal administration.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

### Equivalents

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance which can be adapted to the practice of this invention in its various embodiments and equivalents thereof.

### Examples

### Results

To express the HBsAg particles, the Hepatitis B virus isolate GZ-DYH (adw2 subtype, genotype B2, Genbank ID DQ448619) was identified for gene expression. Based on the protein sequence, we optimized the sequence of the coding DNA for mammalian protein expression. The internal cis-element driving S and M protein expression was removed. DNA fragments containing open reading frames for L, M, and S proteins were synthesized and subcloned into separate mammalian expression vectors.

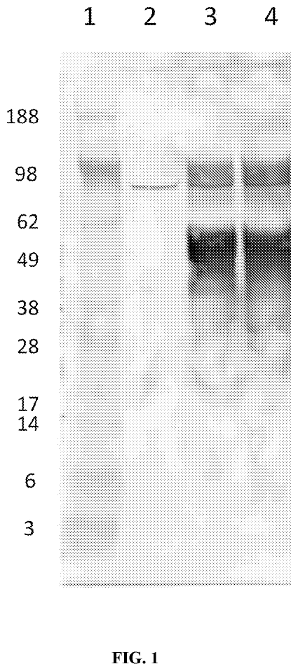

Wildtype coding sequences for L protein contain an internal cis-element that responds to accumulation of L in the endoplasmic reticulum; this cis-element participates in tight regulation of the expression of the L, M, and S proteins. This control mechanism results in differential expression of the surface proteins, S being the most abundant of them, while M and L are expressed at much lower levels of about 5-15 mole % and 1-2 mole %, respectively. There had been no report that analyzes the expression ratio of the L protein by separate expression vectors. Expression of the L-form alone led to secreted forms that are larger than the traditionally reported 42 DKa L protein (Lane 2, FIG. 1). To facilitate secretion of the L-form protein, a secretion signal was added to the N-terminus of the L-protein, and the secreted form (Lane 3, FIG. 1) showed similar glycosylation pattern as the native protein, indicating the L-protein alone underwent complex glycosylation in the Golgi apparatus. However, L expressed alone showed little quantities in the conditioned medium and did not form particles under electron microscopic observation (not shown).

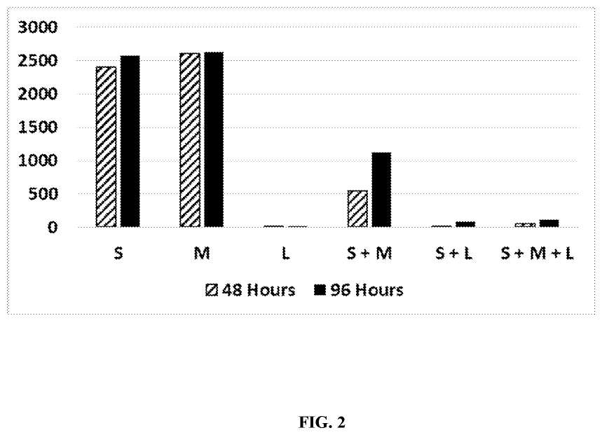

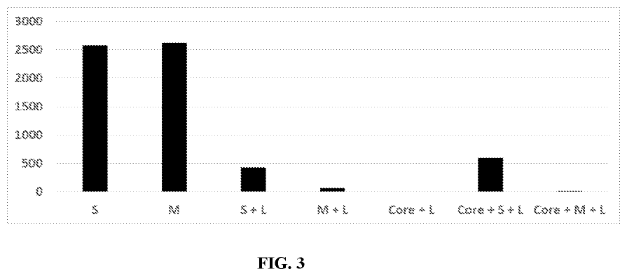

It was found that L-form expression is dependent upon the expression of the S-form and M-form, or only in the presence of the S-form or M-form, the L-form is secreted. Further, expression of the S-form is inhibited by the presence of the L-form (FIGS. 2, 3). The dependence of L-form secretion upon the S-form and M-form suggested that L is assembled into a structure that was driven by the folding of the S or M, although the exact mechanism is not understood. However this feature can be utilized to identify expression clones that accumulate the S- and L-form proteins.

Earlier strategies to produce the HBsAg particles containing all three forms utilized cis-element within the HBsAg coding sequences to drive the expression of the S and M. Given that the promoter strengths of these cis-element is defined, it is not possible to screen for clones that might express the L, M, and S forms with varied ratios. It was found that HBV-particles produced by using a combination of expression vectors had variable compositions of the L, M, and S, opening the possibility to select for clones that may stably express L-form HBV-particles in varied ratios.

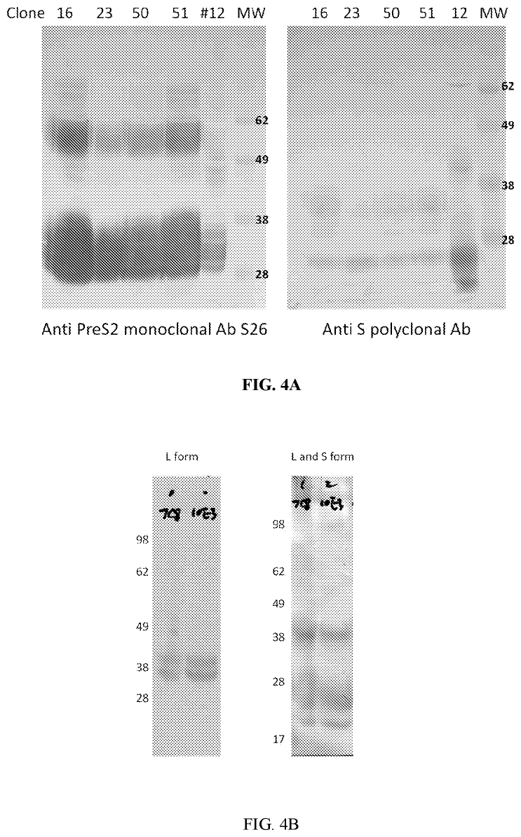

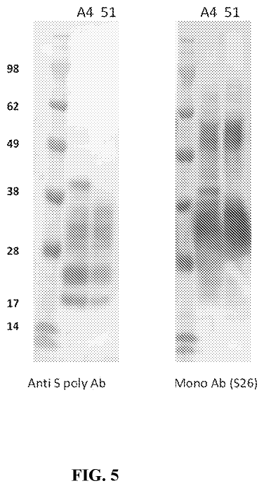

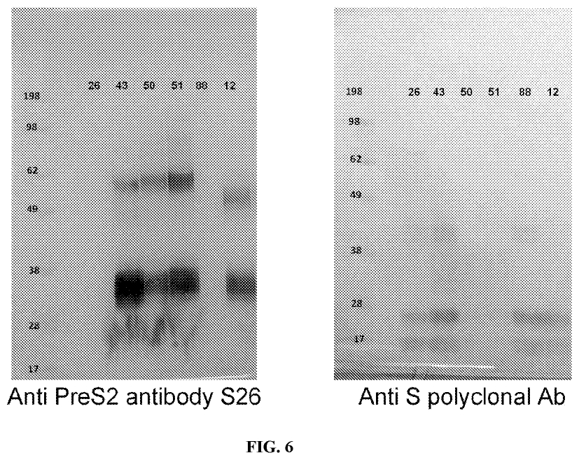

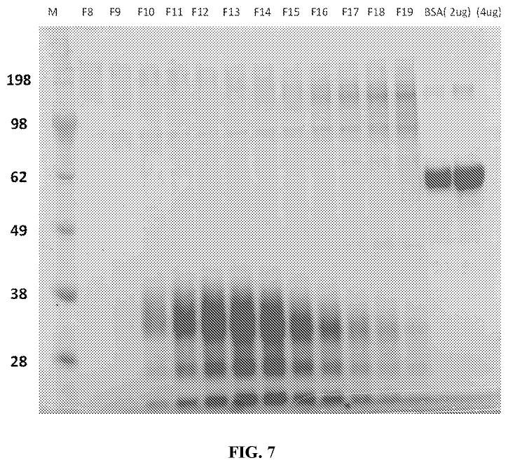

A screen was conducted for several hundred clones derived from 293F that positively expressed HBV particles. The positive clones were selected based on antibodies that recognized three dimensional epitopes in the S. Most of these clones only expressed S protein. Individual clones were screened by Western blotting using anti L-form antibodies, followed by anti-S antibodies. Clone 16 and clone 51 showed expression of S-form at approximately 26 KDa, and additional proteins ranging from 30-38KDa and 51-60KDa that contained epitope for the PreS2 antibody (FIG. 4A). The heterogeneity of the glycosylation suggested that the proteins containing PreS2 epitope underwent complex glycosylation in the Golgi apparatus. Clone 12 and clone A4 contained strong signals for S-form protein at 26KDa and 22KDa, in addition to 42 KDa and 39KDa proteins that were recognized by anti PreS2 antibodies (FIG. 5). The same expression strategies were also applied using CHO expression host. Similarly, clones 7C8, 10E3 derived from CHO cells expressed 26KDa and 22KDa S proteins (FIG. 4B, right panel), and 42 KDa and 39KDa proteins that were recognized by anti PreS2 antibodies (FIG. 4B, left panel). In CHO expression system, the upper 42 KDa L-protein is also recognized by the anti-S polyclonal antibodies. In both expression systems, the 42 KDa and 39 KDa protein bands corresponding to the L-forms showed distinct, sharper bands, suggesting these proteins did not undergo complex glycosylation in the Golgi apparatus. One additional clone, 43, also showed detection of the 42 KDa and 39 KDa protein bands, similar to clone A4 (FIG. 6). To summarize, clone 16 and clone 51 may represent formation of particles at the plasma membranes, while the secretion of the clone 12 and A4 HBsAg particles showed a unique mechanism which involved budding off from the endoplasmic reticulum (Patient, R., Hourioux, C., Sizaret, P. Y., Trassard, S., Sureau, C. and Roingeard, P., 2007. Hepatitis B virus subviral envelope particle morphogenesis and intracellular trafficking. Journal of virology, 81(8), pp. 3842-3851). Both clones 16 and 51 contained a significant amount of proteins in the range of 30-38 KDa which was recognized anti PreS1 antibodies (FIG. 4A), suggesting that the L-form proteins maybe present in significant amount. Purified clone 51 particles contained more 30-38KDa proteins (FIG. 7).

Taken together, combination expression strategy in both 293F and CHO expression systems created HBsAg particles that were not previously characterized and could be used for production of HBsAg particles with desired L, M, and S ratios. Clones that showed significant amount of L and S expressions were selected for scale-up and further characterization.

### Clone 16, 51, A4, Production Scale Up, Purification and Protein Characterization

Stable cell lines 16, 51, and A4 expressed S-form proteins, and in addition L-form proteins that was detected by a PreS2-specific antibody S26. Cells were grown in 293 FreeStyle expression medium (Thermo Fisher) in shake flask cultures. After 72 or 96 hours growth, conditioned medium was harvested and cell debris was removed by centrifugation and filtration. Virus-like particles were purified with two consecutive size exclusion chromatography using Sephacryl 400 resin, or a combination of hydroxy apatite adsorption followed by size exclusion chromatography.

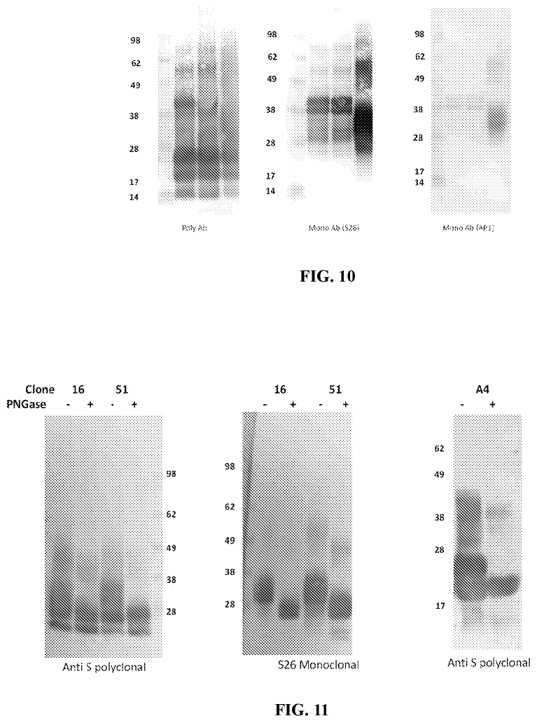

Clone A4 purified protein particles contained two L proteins at 38 KDa and 42 KDa, respectively (FIG. 8, 9). Upon PNGase treatment, the 42 KDa protein was reduced to 38 KDa (FIG. 11), confirming that the 42 KDa is the glycosylated form of 38 KDa. The identities of the L proteins were verified by an antibody raised specifically against a PreS1 antibody AP1 (FIG. 10), as well as by peptide mass spectroscopy (Table 1). In addition, two S proteins were expressed, as represented by 27 and 24 KDa proteins (FIG. 10). Both S proteins were verified by N-terminal sequencing (not shown) and peptide mass spectroscopy (Table 1). Unlike clone 51, the A4 L proteins migrated in the SDS PAGE as distinct bands, suggesting that the protein glycosylation was not further modified in Golgi apparatus and mimicked in vivo particle assembly that occurred in the endoplasmic reticulum.

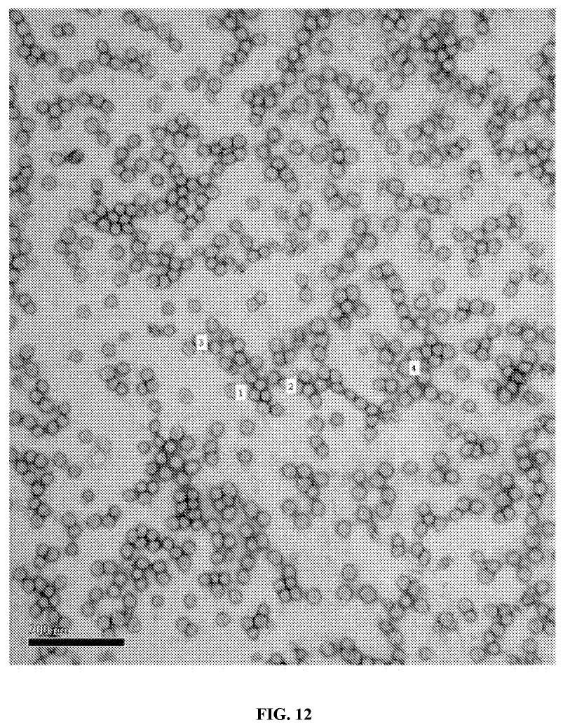

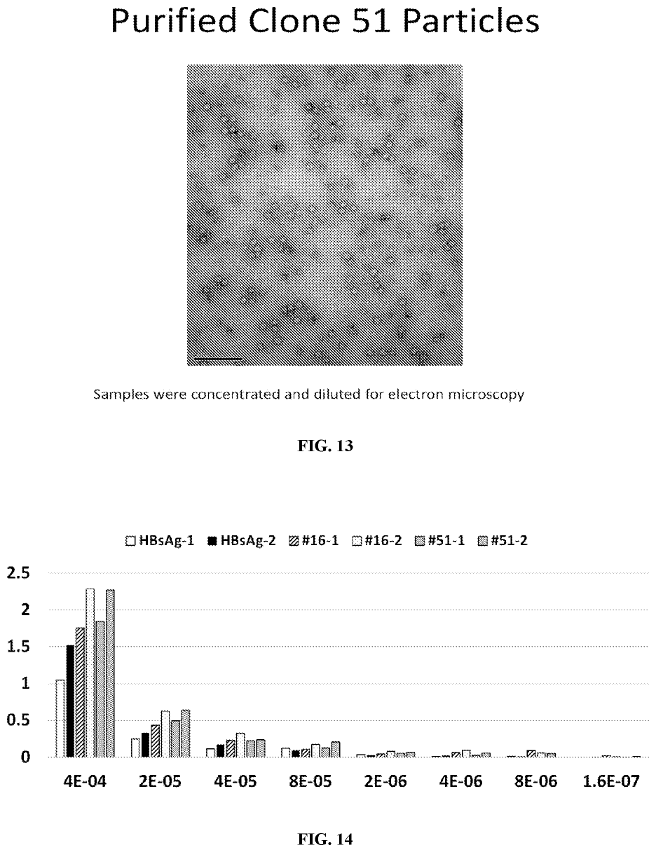

Clone 51 purified protein particles contained L and M protein between 28KDa and 38KDa molecular weight markers. Detection of the protein in this range by PreS1-specific antibody AP1 (FIG. 10) suggested that at least part of the protein mixture in this range is the L protein, albeit with smaller molecular weight than expected. Upon PNGase treatment, the L/M protein mixture was reduced to discrete bands at 28 KDa molecular weight marker. In addition, peptide mapping by mass spectroscopy indicated that part of the protein mixture in this range contained PreS2 sequence (Table 1). The purified proteins from clone 16 and clone 51 formed particles (FIGS. 12, 13), however, more detailed analysis is needed to resolve the L and M protein for the particles.

Both clone A4 and 51 contained S proteins of 27KDa and 24 KDa (FIG. 10). PNGase treatment reduced the 27 KDa S protein band to 24 KDa, confirming that the 27 KDa is the glycosylated form of the 24 KDa S protein (FIG. 11).

As indicated by the SDS PAGE followed by Coomassie staining (FIG. 10), the L protein in the purified HBsAg particles of both A4 and 51 exceeded 10% of the total protein.

TABLE 1
Mass spectroscopy confirmation of peptides
(Peptides identified by mass comparison were listed).

| Protein | Peptide sequences confirmed by mass spectroscopy |
|---|---|
| Clone A4, L42 | ANSEN PDWDLNPHKD NWPDANK |
| | QPTPLSP PLRDTHPQAM QWNSTTFHQT LQDPR |
| | YLWEWA SVR |
| Clone A4, L38 | ANSEN PDWDLNPHKD NWPDANK |
| | QSGRQPTPLSP PLRDTHPQAM QWNSTTFHQT LQDPR |
| | ALY FPAGGSSSGT VSPAQNTVSA ISSILSK |
| | YLWEWA SVR |
| Clone 51, L30 | ALY FPAGGSSSGT VSPAQNTVSA ISSILSK |
| | YLWEWA SVR |
| Clone A4, S27 | YLWEWA SVR |
| Clone A4, S24 | YLWEWA SVR |
| Clone 51, S27 | YLWEWA SVR |
| Clone 51, S24 | YLWEWA SVR |

### Immunization of Clone 16 and 51 Produced Proteins

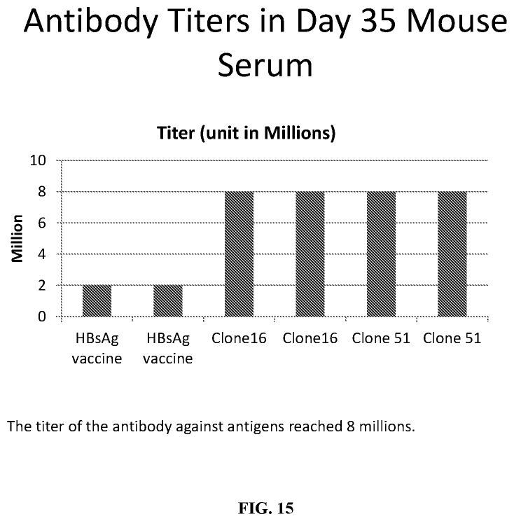

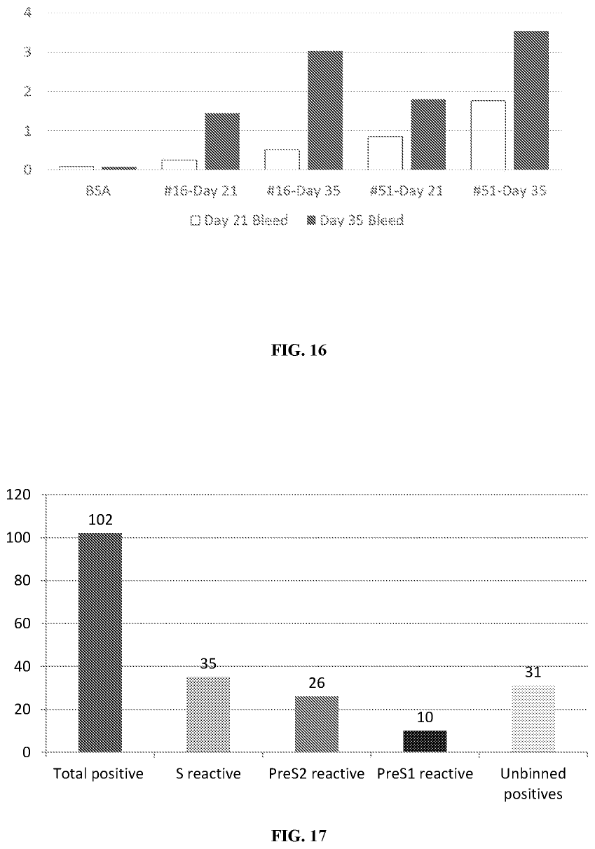

Two mice each were immunized using purified LMS virus-like particles derived from clone 16 and clone 51, respectively. Yeast-derived HBsAg was used injected into two mice as reference. For all experiment, mouse strain Balb C was used. A boost injection was given to all the mice after 14 days. Blood was drawn from the mice 35 days after the initial immunization, and the titers of the antibody response against the virus-like particles were determined by serial dilutions (FIG. 14). Final dilutions that showed significant responses as compared to the background was determined as the titer. Antibody titer for yeast-derived HBsAg was 2e6, for LMS HBsAg was 8e6 (FIGS. 14, 15). In summary, immunization using clone 16 and clone 51 HBsAg particles generated approximately four fold higher antibody titers compared to that obtained by using yeast-based antigens (FIG. 15). To characterize the immune responses against the HBsAg proteins, the purified HBsAg proteins, yeast-derived S antigen, and E. coli-derived PreS1, PreS2 peptide sequences were used. These antigens were coated to polystyrene 96-well microtiter plates, and serial dilutions of the sera were incubated followed by detection of the bound mouse IgG1 antibodies. The strongest immune response was against the S antigen, followed by PreS2 and PreS1 antigens (FIG. 16).

In order to understand which epitope was responsible for eliciting immune responses in mice, hybridomas were generated using spleen cells from the immunized mice. In total 102 hybridomas were found to react against the purified HBsAg particles (FIG. 17). Using various antigen to categorize the hybridomas, we found that antibodies from 35 hybridomas reacted against the S antigen, and 26 and 10 hybridomas reacted against the PreS2 and PreS1 peptides, respectively (FIG. 17). Further, 31 hybridomas were reactive against the purified HBsAg particles but were not reactive against S, or the linear peptide antigens derived from the PreS1 and PreS2 sequences, suggesting that these antibodies may recognize non-linear epitopes present in the PreS regions of the purified HBsAg antigens. However, the PreS2 and PreS1 peptide antigens used to analyze the antibody titers in the sera lacked secondary or tertiary structures, and the responses against the three dimensional epitope in the PreS region could not be delineated.

### Materials and Methods

### Cloning and Cell Line Selection

The coding sequences of the HBV surface antigens were based on the Hepatitis B virus isolate GZ-DYH (Genbank accession number DQ448619, serotype adw2). For protein expression, the open reading frames were codon-optimized for mammalian expression systems. The internal cis-elements such as the promoters for transcription initiation of M and S forms were abrogated by silent substitutions. Genes encoding L, M, and S forms were synthesized separately at Genewiz (South Plainfield, N.J.), and the DNA fragments were subcloned into expression vectors, respectively. The expression plasmid constructs were transfected into HEK293 cells that were previously adapted to serum-free growth. Stable expression cell lines were selected by single cell cloning in 96-well culture plates using a flow cytometer. Approximately 10% single cells yield cell lines and give rise to expression clones. Expression clones were selected based on ELISA screening followed by Western blot analysis using antibodies specific to the PreS2 (NovusBio, Littleton, Colo.), PreS1 (ProspecBio, East Brunswick, N.J.), and HBsAg S proteins (Creative Diagnostics, Shirley, N.Y.).

FreeStyle™ 293 Expression Medium (Thermo Fisher Scientific, Waltham, Mass.) was used for all cell line cloning and scale up procedures. Western blot analysis of the recombinant HBV surface antigen was carried out using anti PreS2 monoclonal antibody S26, anti PreS1 monoclonal antibodies AP1, AP2 (Santa Cruz Biotechnology, Dallas, Tex.), and rabbit anti HBsAg S polyclonal antibodies (Fitzgerald Industries International, Acton, Mass.). The HBsAg ELISA kit was obtained from Creative Diagnostics (Shirley, N.Y.).

### Production of HBsAg Particles

Shake flask cultures were used for small scale production for the HBsAg particles. Conditioned medium of stably transfected cell lines were harvested, and the HBV virus-like particles were purified through a combination of tangential flow filtration and concentration, hydroxy apatite adsorption size exclusion chromatography, and anion exchange chromatography. The morphology of the purified particles were visualized using electron-scanning microscopy. Protein compositions of the purified HBsAg particles were analyzed by silver staining and Western blot, or Coomassie blue staining followed by tryptic digestion and peptide mass spectroscopy. Protein concentration was determined by BCA method.

Deglycosylation of HBV surface antigens by PNGase treatment were carried out according to the manufacturer's specifications (New England Biolab, Ipswich, Mass.).

For N-terminal sequencing, proteins were separated by SDS PAGE and transferred to PVDF membrane by Western blotting procedure. PVDF membranes were stained with Coomassie Blue, protein bands were sliced out and subjected to Edman degradation followed by HPLC analysis.

Reverse phase HPLC is carried out using Vydac 214TP C4 column (10 um, 4.6×150 mm). The mobile phase is acetonitrile/H2O 20% to 80% gradient at a flow rate 1 ml/min.

### Peptide Mapping by Mass Spectroscopy

Purified proteins were separated by SDS PAGE, followed by Coomassie Blue staining. Protein bands were sliced out and gel slices were used for in-gel trypsin digestion followed by LC-MS analysis. Peptide masses were compared to known peptide sequences in databases and identification was confirmed by mass comparison. The peptides identified by mass spectroscopy are listed in Table 1.

### Immunization of Mice and Determination of Antibody Responses

Mice of Balb C strain were purchased from Charles River Laboratories. Mice were grouped in two per group. 10 μg Yeast-derived HBsAg, or 10 μg of purified proteins from clone #16 or #51 were injected after mixing with aluminum adjuvant, followed by boost injections after 14 days. Blood was drawn from the mice 35 days after the initial immunization, and the titers of the antibody response against the virus-like particles were determined by serial dilutions (FIG. 16). Antibody titers were determined by using yeast-derived S antigen, PreS1 peptide, PreS2 peptide, or purified LMS HBsAg coated to Hi-Binding 96 well assay plates. Student t-test was used to determine the significance of antibody titers.

### Hybridomas and Epitope Binning

Spleens from the immunized mice were removed one day after the final injections. Spleen cells were isolated and fused to murine myeloma cells according to standard procedures. Hybridoma clones were tested for reactivity against the purified HBsAg particle containing the PreS regions (FIG. 17). To determine the regions of the epitope, yeast-based HBsAg S, PreS1 peptide, and PreS2 peptide were coated to polystyrene 96-well microtiter plates. Conditioned medium of the hybridomas were incubated with bound antigens followed by anti-mouse IgG detection.

1. A hepatitis B virus (HBV) vaccine particle, comprising a recombinant HBV surface antigen comprising:
L surface protein;
optionally M surface protein; and
optionally S surface protein;
wherein the percentage of L surface protein in the L, M, and S surface proteins is at least about 1 mole %.

2. The HBV vaccine particle of claim 1, wherein the percentage of L surface protein in the L, M, and S surface proteins is at least about 2 mole %, 3 mole %, 4 mole %, 5 mole %, 6 mole %, 7 mole %, or 8 mole %.

3. The HBV vaccine particle of claim 1, wherein the percentage of L surface protein in the L, M, and S surface proteins is more than about 8 mole %.

4. The HBV vaccine particle of claim 1, wherein the percentage of L surface protein in the L, M, and S surface proteins is more than about 9 mole %, 10 mole %, 11 mole %, 12 mole %, 13 mole %, 14 mole %, 15 mole %, 16 mole %, 17 mole %, 18 mole %, 19 mole %, 20 mole %, 21 mole %, 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, or 50 mole %.

5. The HBV vaccine particle of claim 1, wherein the percentage of L surface protein in the L, M, and S surface proteins is at least about 60 mole %, 70 mole %, 80 mole %, 90 mole %, or 100 mole %.

6. The HBV vaccine particle of claim 1, wherein the HBV vaccine particle does not include M or S protein.

7. The HBV vaccine particle of claim 1, wherein the HBV vaccine particle is a virus-like-particle.

8. The HBV vaccine particle of claim 1, wherein the percentage of L surface protein in the L, M, and S surface proteins is from about 10 mole % to about 40 mole %, 5-15 mole %, 15-25 mole %, 25-40 mole %, or 40-60 mole %.

9. The HBV vaccine particle of claim 1, wherein the L surface protein is encoded by a recombinant nucleic acid sequence which does not have an internal cis-element.

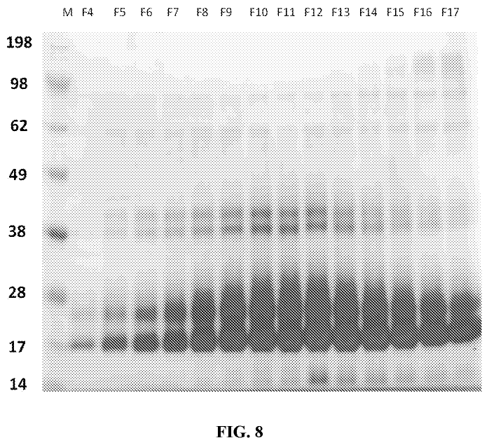

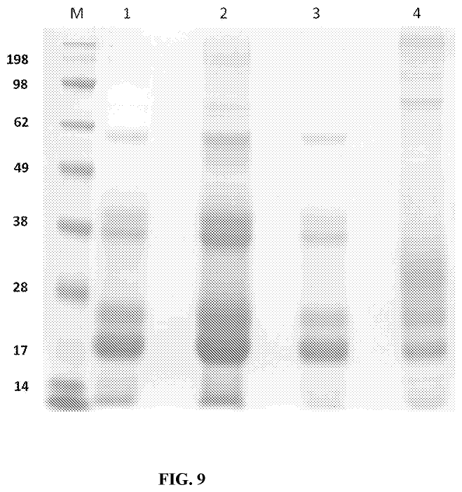

10. The HBV vaccine particle of claim 1, comprises clone A4 or 51 as shown in FIG. 9.

11. A HBV vaccine comprising the HBV vaccine particle of claim 1 and an adjuvant.

12. The HBV vaccine of claim 11, wherein the adjuvant is selected from the group consisting of alum, a toll-like receptor, and colloidal gold.

13. A method of treating or preventing HBV infection in a subject in need thereof, comprising administering to the subject an effective amount of the HBV vaccine of claim 11.

14. The method of claim 13, wherein the subject is human.

15. A recombinant nucleic acid sequence encoding L surface protein, wherein the recombinant nucleic acid sequence does not have an internal cis-element.

16. A recombinant expression vector for expressing L surface protein, comprising the recombinant nucleic acid sequence of claim 15.

17. A cell transformed with the recombinant expression vector of claim 16.

18. The cell of claim 17, where the cell is additionally transformed by
a second recombinant expression vector comprising a second recombinant nucleic acid sequence encoding the S surface protein, and
a third recombinant expression vector comprising a third recombinant nucleic acid sequence encoding the M surface protein.

19. The cell of claim 17, wherein the cell is additionally transformed by one or more additional recombinant expression vectors.

20. The cell of claim 17, wherein the cell is additionally transformed by a fourth expression vector comprising a fourth recombinant nucleic acid sequence encoding the HBV core antigen.

21. The cell of claim 17 derived from E coli, fungus, an insect or mammalian protein expression host.

22. The cell of claim 21 derived from a HEK-293 cell or a CHO cell.

23. A method for preparing an HBV vaccine particle, comprising:
a) providing recombinant expression vectors comprising a first, second, and third recombinant nucleic acid sequences encoding L, M, and S surface proteins respectively; and wherein the first, second, and third recombinant nucleic acid sequences do not have an internal cis-element;
b) transforming cells with the recombinant expression vectors; and
c) culturing and selecting for cells to co-express L, M, and S surface proteins.

24. The method of claim 23, wherein each of L, M, and S surface proteins is in a separate expression vector.

25. The method of claim 23, further comprising selecting for cells to express L surface protein in a percentage of at least about 1 mole %, 2 mole %, 3 mole %, 4 mole %, 5 mole %, 6 mole %, 7 mole %, 8 mole %, 9 mole %, 10 mole %, 11 mole %, 12 mole %, 13 mole %, 14 mole %, 15 mole %, 16 mole %, 17 mole %, 18 mole %, 19 mole %, 20 mole %, 21 mole %, 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, or 50 mole % in the L, M, and S surface proteins.

26. The method of claim 23, further comprising selecting for cells to express L surface protein in a percentage of at least about 60 mole %, 70 mole %, 80 mole %, 90 mole %, or 100 mole % in the L, M, and S surface proteins.

27. The method of claim 23, wherein the recombinant expression vectors further comprises a fourth recombinant nucleic acid sequence encoding the HBV core antigen; and step c) comprises culturing and selecting for cells to co-express L, M, and S surface proteins and HBV core antigen.

28. The method of claim 23, wherein the cells are derived from an insect or mammalian protein expression host.

29. The method of claim 28, wherein the cells are derived from HEK-293 cells or CHO cells.
